(19) United States
(12) Patent Application Publication
(10) Pub. No.: US 2005/0106103 A1
(43) Pub. Date: May 19, 2005

(54) Method and system for characterizing tactile perception

(75) Inventors: Anne Dussaud, New York, NY (US); Junqi Ding, Tenafly, NJ (US); Alexander Lips, Edgewater, NJ (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 10/712,490

(22) Filed: Nov. 13, 2003

(51) Int. Cl. A61K 7/00; A61B 5/00

(52) U.S. Cl. 424/9.2; 600/300; 600/587; 424/401

(57) ABSTRACT

An acoustic emission system for objectively measuring tactile skin attributes and methods of using same. The system includes: (A) Means for generating an acoustic emission signal from skin; (B) Means for collecting, storing and displaying said emission signal; (C) means for correlating said emission signal with an attribute of said skin; wherein said system is used as a clinical tool to evaluate efficacy of cosmetic skin care and/or cleansing products. A cosmetic product selection system is also provided which includes a cosmetic composition for reducing the appearance of undesirable skin attributes and an acoustic emission system associated with the composition. A method for assessing tactile skin attributes using an acoustic emission system, as well as of evaluating progress of the combat against the signs of undesirable skin attributes occurring over a period of time within which the composition is applied to an area of skin being monitored is provided.

### FIELD OF THE INVENTION

The invention concerns a method and system for characterizing tactile perception on skin using acoustic emission, as well as methods of using the system for demonstrating proof of efficacy and/or facilitating cosmetic product selection.

### BACKGROUND OF THE INVENTION

The ultimate goal of any cosmetic product or method, is a satisfied consumer. Many cosmetic products, either leave-on and/or wash off products, advertise a variety of skin benefits. While expert graders may be trained to use diagnostic equipment or to perceive the difference, consumers usually cannot easily discern whether the claimed benefit is actually delivered, or a quantitative extent to which it is delivered. Skin conditions are typically evaluated subjectively through the senses, particularly through sight and touch. An objective measure of the physical parameters controlling the key attributes would provide a useful tool in the characterization of sensory attributes.

Among skin diagnostic techniques, acoustic emission has not been commonly used for skin characterization. Acoustic emission has been commonly used in the field of mechanical systems and musical instruments. It has also been used in the field of fabric sensory, since for consumers, the sound of fabrics is part of the sensory experience.

Tanaka, M. et al., “The ‘Haptic Finger’—a new device for monitoring skin condition,” Skin Research and Technology, 9:131-136 (2003) describe a device simulating a finger, formed of several layers of materials (stainless steel plate, sponge rubber, a piezopolymer film, acetate film and gauze). This artificial finger is rubbed on skin as it records the mechanical stresses generated in flexible piezopolymer films.

Flament, F., et al., “Finger perception metrology. Correlation between friction force and acoustic emission,” Abstract of a presentation at a skin conference in Hamburg, 2003, describe a method where the finger of a subject is held by a motorized support which moves the finger over a surface of materials where the friction force and the acoustic emission signals are detected.

WO 02/24071 A2 relates to a method, apparatus and system for assessing hair condition by measuring friction in hair samples. A friction member, similar to a “comb” generates friction at the contact between the comb and the hair. The frictional noise generated by the forces during comb motion is captured by a frictional noise sensor.

A need remains for a tool and method for the objective measurement of tactile attributes of human skin.

### SUMMARY OF THE INVENTION

Accordingly, Applicants have developed an acoustic emissions system and method for objective assessment of skin condition before, during, and after application and/or wash-off of products onto or from skin. The acoustic emission measurement system includes:
(A) Means for generating an acoustic emission signal from skin;
(B) Means for collecting, storing and displaying said emission signal; and
(C) means for correlating said emission signal with an attribute of the skin.

The inventive system and method can be used:
(1) as a clinical tool to evaluate the efficacy and/or tactile perception on skin of cosmetic skin care and/or cleansing products, i.e., from a clinical and/or consumer perspective;
(2) as a consumer communication tool to determine the degree of change that is meaningful and ideal to the consumer, i.e., to define the distribution of skin attributes in a specific population and/or to set technical and consumer targets; and
(3) as a point of purchase diagnostic device to allow a consumer a simple method to evaluate before and after treatment changes in skin condition, thereby facilitating product screening and selection, product customization and/or compliance with a product usage regimen.

The system and method can be used by the consumer directly, but is preferably applied by a beautician, clinician, sales associate, or other professional adviser.

The very sensitive inventive method detects acoustic signal during touching to assess the in-use sensory performance of personal care products and extract specific sensory attributes or sensory profile. In another aspect of the present invention, the sensory attributes or profile can be linked to consumer language and/or communicated to consumers. Communication media may include the Internet, camera, palm pilot, mobile phone; mobile camera phone, advertising and promotional material, including television, magazines, brochures, posters, flyers, and hand-outs; and/or water-insoluble substrate. The system and method can be used to support claims about various benefits from skin care products, such as moisturization, rinsability of cleansers and a wide range of sensory benefits (i.e smoothness, silkiness).

A cosmetic product selection and/or customization system is provided which includes:
(i) at least one cosmetic composition for reducing the appearance of at least one undesirable skin attribute; and
(ii) an acoustic emission system associated with said cosmetic composition; the acoustic emission system having a means for evaluating current appearance of skin attributes or progress in reducing the appearance of said undesirable attributes with the use of said cosmetic composition.

Further, there is provided a method for evaluating attributes an area of facial skin and/or the efficacy of a cosmetic product for reducing the appearance of an undesired attribute, including:
(A) providing an acoustic emission system;
(B) applying the cosmetic product to an area of skin;
(C) generating acoustic emission data for said area of skin;
(D) analyzing said data to assess said skin attributes; and
(E) repeating steps (C) and (D) at a future time followed by comparison of data resultant from first and second assessments of the skin.

### BRIEF DESCRIPTION OF THE DRAWINGS

Although not limited thereto, additional objects, features and benefits of the present invention will become more readily apparent from consideration of the drawings in which:

FIG. 1 is a schematic diagram of an acoustic emission system according to the present invention;

FIG. 2 shows acoustic profiles of SLES/water solution at three different concentrations, generated using the system of FIG. 1;

FIG. 3 shows an acoustic profile of a wash-off (cleansing) product obtained with the system of FIG. 1, wherein a hydrophone and accelerometers are used simultaneously;

FIG. 4 shows acoustic profiles of two different cleansing products obtained using the same system as that used to generate FIG. 3, wherein (a) and (b) are the acoustic signals (sound pressure and acceleration, respectively) from product A; (c) and (d) are acoustic signals (sound pressure and acceleration, respectively) from product B;

FIG. 5 shows acceleration signals of rubbing on a leave-on product over a time period of 9 minutes, obtained using the system of FIG. 1 wherein two accelerometers and two microphones are used;

FIG. 6. Shows acceleration signals of two different products over a certain time period of application, obtained using the same system as that used to generate FIG. 5;

FIG. 7 shows acceleration signals of after-feel of two cleansing products, with measurements taken after a period of towel drying, wherein FIG. 7(a) shows the dry feel after using a harsh cleansing product; and FIG. 7(b) shows the moisture feel after using a mild cleansing product.

### DETAILED DESCRIPTION OF THE INVENTION

Now consumers have been provided with a system and method that meets the need for objective assessment of skin and application and/or wash-off of products onto or from skin.

The present invention is based on a very sensitive method, which detects acoustic signal during touching to assess the in-use sensory performance of personal care products and extract specific sensory attributes or sensory profile, which can be linked to the consumer language and/or communicated to consumers. It can be used to support claims about various benefits from skin care products, such as moisturization, rinsability of cleansers and a wide range of sensory benefits (i.e smoothness, silkiness).

The inventive system and method can be used:
(1) as a clinical tool to evaluate the efficacy of cosmetic skin care and/or cleansing products, both from a clinical and consumer perspective;
(2) as a consumer communication tool to determine the degree of change that is meaningful and ideal to the consumer, i.e., to define the distribution of skin attributes in a specific population and/or to set technical and consumer targets; and
(3) as a point of purchase diagnostic device to allow a consumer a simple method to evaluate before and after treatment changes in skin condition, thereby supporting claims of product benefits and affecting/influencing product selection, product customization and/or compliance with a product usage regimen.

### Acoustic Emission System

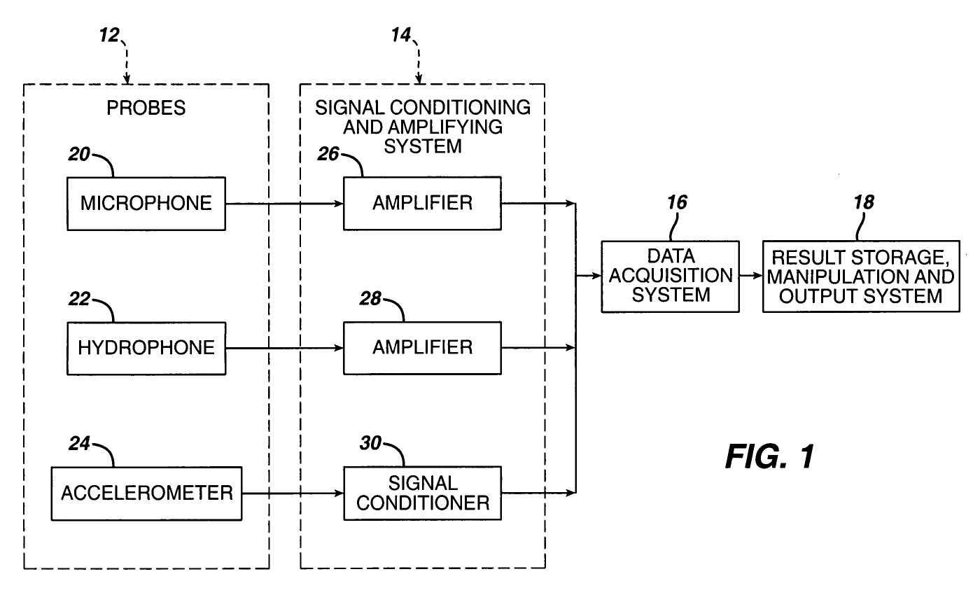

With reference to FIG. 1, a schematic diagram of an acoustic emission system 10 according to the present invention is shown. Acoustic emission system 10 includes probe(s) 12 that operate in an acoustic medium to pick up a signal from sound or vibration generated by a substrate (not shown), such as biological tissues, skin tissues, or hard surfaces. Probe(s) 12 are in communication with a signal conditioning and amplifying system 14, connected to a data acquisition system 16, which, in turn, is connected to a result storage, manipulation, and output system 18. A power source (not shown) is also provided for powering system components. Each of the individual components may be commercially available or custom built, and any of the components 14, 16, 18 may be combined or eliminated, such as, for example if analog signal is stored in that form, or if recording is directly to a tape recorder or CD or DVD.

Acoustic Medium

The acoustic medium may be gas or liquid. Generally, in practice, the acoustic medium is air, water, or aqueous solution.

Acoustic Probe(s)

System 10 has at least one acoustic sensor, or probe 12. Probe 12 may comprise at least one microphone 20, for gaseous medium, or at least one hydrophone 22, for aqueous medium. Microphone 20 and/or hydrophone 22 may be used individually, in combination with each other, and/or in combination with a vibration sensor, or accelerometer 24. One or more accelerometers 24 may be used individually, in combination with each other, or in combination with at least one microphone 20 and/or hydrophone 22.

Signal Conditioning System

Signal conditioning system 14 is in communication with probe(s) 12 and may be comprised of at least one amplifier 26, 28 to amplify sound received by microphone 20 and/or hydrophone 22. A signal conditioner 30 is provided to manipulate signal received from accelerometer 24.

Data Acquisition System

The data received by any one or more of amplifiers 26, 28 and/or accelerometer 24 is transferred to data acquisition system 16. In the alternative, data may be transferred to data acquisition system 16 directly from probe(s) 12.

Result Storage, Manipulation and Output System

Data from data acquisition system 16 is transferred to result storage, manipulation and output system 18. In alternative embodiments, result storage, manipulation and output system 18 may receive data directly from probe(s) 12 and/or signal conditioning system 14.

System 18 may be a personal computer or similar digital data storage medium, such as a hand-held device (e.g. Palm Pilot, cellular phone), magnetic tape, CD, or DVD. At this point, the data may be represented in graphical form and printed, in a variety of communication media. Communication media may include the Internet, camera, palm pilot, mobile phone; mobile camera phone, advertising and promotional material, including television, magazines, brochures, posters, flyers, and hand-outs; and/or water-insoluble substrate. Additionally, correlations between skin condition as data received qualitatively from consumers and data received from acoustic emission system 10 may be made and/or may reside within the result storage, manipulation and output system 18.

System 10 and method can be used to support claims about various benefits from skin care products, such as moisturization, rinsability of cleansers and a wide range of sensory benefits (i.e smoothness, silkiness).

### Acoustic Emission Method

The inventive method uses acoustic signals emitted generated from contact with a substrate, preferably skin, when the skin on one area of the human body slides on the skin on another area of the body, i.e., skin on skin. The frictional forces in the skin/skin contact generate vibration patterns that are sensed by probe(s) 12 placed near the skin/skin contact area and recorded by result storage, manipulation, and output system 18. Advantageously, probe(s) 12 do not affect the contact between one area of skin and another area of skin, since it can detect vibrations near the contact area without interfering with the contact. Therefore according to the present invention, the acoustic signal reflects accurately the skin/skin contact properties and the in-use conditions.

Acoustic emission is recorded during the gentle rub of the hand or finger on another skin part. It is typically detected on the forearm, the hand or the face, but could also be used for other body parts. The acoustic emission signals can be recorded by several kinds of probe(s) 12. Microphones 20 or hydrophones 22 can be mounted close to the skin/skin contact, but do not touch the skin. For these acoustic sensors, air or water, respectively, is medium, or the vehicle of the vibration produced on the skin. Small or miniature vibration sensors (i.e. accelerometers 24) can be mounted to the skin, near the area of contact. In that case, the vibrations generated by the rubbing are transmitted by the skin tissues. Acoustic sensors may be commercially available devices or custom made devices. The acoustic emission signal is conditioned and/or amplified, by amplifiers 26, 28 and/or signal conditioner 30, respectively, and sent to data acquisition system 16. Data from data acquisition system 16 is then sent to storage, manipulation, and output system 18, such as a PC sound card or other appropriate digitizing device where sound emission data may be saved and displayed. Various standard signal analysis methods can be applied to the signal, and custom methods may be developed.

In typical use, the hand rubs the forearm in a regular manner or the two fingers are rubbed on each other. Since the acoustic emission depends on the speed of the rubbing and the pressure at the skin/skin contact, several recordings are performed for a given subject in order to define a control vibration pattern. The root mean square of the signal recorded other a given period of time can be used to assess the consistency of the rubbing process. The acoustic emission signals are then monitored during the application of products or at intermittent intervals after application of a product.

Signal Analysis

Applicants have found that the amplitude of the vibrations and the frequency content of the acoustic signal, or waves, monitored as a function of time are very sensitive to the change of the skin/skin contact properties associated with the use of products.

The signal wave can be associated with, or correlated with, particular sensory properties. For example, analysis of these signals allows to characterize the rinsability and “feel” or tactile perception of skin cleansers, the tactile feel after use of cleansers, and the tactile feel resulting from the application of cosmetic creams. Acoustic signal wave analysis can also be used to detect the deposition of moisturizing agents from cosmetic products and cleansers.

Cosmetic Product System

A cosmetic product system is also provided according to the present invention, including a cosmetic composition associated with acoustic emission system 10.

One aspect of the present invention provides a cosmetic product system wherein a cosmetic composition is packaged with an acoustic emissions system 10 in whole or in part, such as with a typical acoustic graph. A variety of packaging arrangements are envisioned. An acoustic graph may be incorporated as a panel segment of a carton, the latter protectively surrounding the cosmetic composition. In a variation thereof, the graph may be detachably joined to the package through a perforated or weakened construction line, or through an adhesive joinder. In another embodiment, the exterior or interior of the package may be imprinted with instructions for a consumer to sample the product with acoustic emission system 10 located at the point of purchase.

Cosmetic Compositions

Cosmetic compositions, such as for reducing the appearance of facial skin pores, wrinkles or other undesired facial attributes, may be in the form of creams, lotions, toners, pastes, sticks (e.g. lipsticks), or powders. These cosmetics normally will include a carrier. Suitable carriers include water, emollients (esters, hydrocarbons, silicones, polyols and mixtures thereof, emulsifiers, thickeners and combinations thereof. Most often the carrier will be an emulsion such as an oil-in-water or water-in-oil type. Amounts of the carrier may range from about 1 to about 99.9% by weight.

Pore reduction active or agents for reducing the appearance of pores may include: astringents, humectants, acne and sebum suppressants, desquamation enhancers, keratolytics, and make-up, among other pore reduction actives known to one skilled in the art.

Astringents

Examples of astringents include, but are not limited to, ethanol, witch hazel, zinc and aluminum salts, and polyphenols.

Humectants

Humectants include propylene glycol (available from Spectrum), glycerol, and sorbitol, among other humectants known to one skilled in the art. Humectants are known as excellent moisturizers for skin, scalp and hair. See for instance U.S. Pat. No. 5,858,340, incorporated by reference herein.

Acne and Sebum Suppressants

Anti-acne actives include benzoyl peroxide and salicylic acid, among other anti-acne agents known to one skilled in the art. Sebum suppressants include compounds of the general formula A:
R—O-M (A)
wherein:
R is a branched alkyl or alkenyl chain having at least 7 carbon atoms, and at least two branches;
O is an oxygen atom; and
M is (—(CH₂)ₚO)ₙ—(CH₂)ₘCO₂X)
   where n is 0 or an integer between 1 and 7, m is an integer between 1 and 4, p is an integer between 2 and 4; and X is hydrogen, a methyl group, an ethyl group, or a cation. The cation is selected from the group consisting of sodium, lithium, potassium, calcium, copper, magnesium, manganese, strontium, sulfur, zinc, and amines. Preferably, X is hydrogen or a cation.
   Make-Up

Examples of make-up useful for reducing the appearance of pores include foundations, moisturizers, foamers, and concealers, among other make-ups known to one skilled in the art.

Anti-aging actives may include retinoids, ceramides, alpha or beta-hydroxycarboxylic acids, flavonoids, vitamins, sunscreens, anti-oxidants, preservatives and mixtures thereof.

Typical retinoids include retinol, retinoic acid and retinol esters. The latter include retinyl palmitate, retinyl linoleate, retinyl propionate, retinyl acetate and retinyl salicylate.

Alpha-hydroxy acids include the free acid, lactone and salt forms of glycolic acid, lactic acid, citric acid, gluconolactone, glucarolactone, tartaric acid, malic acid and mixtures thereof. Beta-hydroxycarboxylic acids are exemplified by salicylic acid as well as its esters (e.g. tridecylsalicylate) and salts including ammonium, alkanolammonium and alkalimetal salts.

Ceramides include Ceramide 1, Ceramide 2, Ceramide 3, Ceramide 3a, Ceramide 3b, Ceramide 4, Ceramide 5 and Ceramide 6, as well as pseudoceramides, phytosphingosines and tetraacetyl phytosphingosine.

Other skin benefit agents may be included as optional components. Vitamins may include ascorbic acid as well as its water-soluble and water-insoluble derivatives. Illustrative are ascorbyl tetraisopalmitate, magnesium ascorbyl phosphate and ascorbyl glucoside. Other vitamins include Vitamin B3 (niacin, niacinamide and panthenol), biotin, folic acid, tocopherol and its esters (e.g. tocopherol isopalmitate, tocopherol acetate), Vitamin D and combinations thereof.

Antioxidants include BHT (butylated hydroxytoluene), BHA (butylated hydroxyanisole), disodium EDTA (available from Ciba), sodium citrate, hydroquinone, ferulic acid and esters thereof, green tea extract, lipoic acid, N-acetyl cysteine, resveratrol and combinations thereof.

Amounts of the pore or wrinkle reduction or other actives may range anywhere from 0.0000001 to 30%, preferably from 0.0001 to 15%, more preferably from 0.1 to 5%, by weight.

Methods of Use

The inventive system, and methods can be used:
(1) as a clinical tool to evaluate the efficacy of cosmetic skin care and/or cleansing products, both from a clinical and consumer perspective;
(2) as a consumer communication tool to determine the degree of change that is meaningful and ideal to the consumer, i.e., to define the distribution of skin attributes in a specific population and/or to set technical and consumer targets; and
(3) as a point of purchase diagnostic device to allow a consumer a simple method to evaluate before and after treatment changes in skin condition, thereby affecting product selection, product customization and/or compliance with a product usage regimen.

The system and method can be used by the consumer directly, but is preferably applied by a beautician or other professional adviser.

Specifically, the system may be used for determining the condition of facial skin pre- and post-treatment, or to track changes in facial attributes, associated with a variety of factors, such as effects of food, activity, menstrual cycle. Pre-treatment acoustic emission system 10 measurements may be used in selecting an appropriate cosmetic product. For example, different product formulations may be recommended depending on the individual skin condition as measured by the inventive acoustic system 10 and method. Acoustic measurements may be represented in a variety of media in association with skin care and/or cleansing products within the scope of the present invention.

Subsequent to a baseline analysis of facial attributes using acoustic emission system 10 and method, treatment is begun with a selected cosmetic product for the particular facial attribute. Treatment is continued for a period of time sufficient to allow the product to treat the signs of the particular facial attribute.

After the treatment period of time, such as four weeks, another acoustic measurement is taken. Testing may occur thereafter at 6, 8, 12, 16 and/or 20 weeks. The time intervals and numbers may be longer or shorter. If the cosmetic product is properly functioning, fewer and/or smaller undesirable facial attributes will appear upon acoustic evaluation. This procedure can then be repeated at six or eight weeks or at any further time interval.

The acoustic system 10 and method may be used in conjunction with a variety of media for displaying measurement results, including in or out of home use of the Internet, webcam, palm pilot, mobile phone, and other media capable of displaying the results in graphical, quantitative, and/or qualitative terms. A strip embodying such result may be given out to consumers at point of sale or at a store display.

An objective clinical grading scale, whereby each image is associated with a number, may be developed.

Except in the operating and comparative examples, or where otherwise explicitly indicated, all numbers in this description indicating amounts of material ought to be understood as modified by the word “about”.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

All parts, percentages and proportions referred to herein and in the appended claims are by weight unless otherwise illustrated.

In the following, several examples of application of the inventive system and method are described. The following is by way of example, not by way of limitation, of the principles of the invention to illustrate the best mode of carrying out the invention.

### EXAMPLE 1

This example uses acoustic measurements to characterize the tactile properties on skin of surfactants applied thereto, e.g. slimy, squeaky and slimy/squeaky transition evaluation, using acoustic measurements.

An acoustic emission system 10 as generally described with reference to FIG. 1 was used for testing squeakiness and its transition properties of surfactant solutions. One probe 12, i.e., hydrophone 22 (Bruel & Kjaer, Atlanta, Ga., 8103 hydrophone), was mounted onto an inner wall of a container (not shown) for surfactant property testing in solution. The inner container wall may be machined with certain roughness in order to reduce the reflection of sound waves from the container wall. The charge signal from hydrophone was conditioned to voltage signal via a Bruel & Kjae Conditioner amplifier 28. Clean fingers were rubbed against each other inside the surfactant solution while data acquisition system 16 (CoolEdit 2000 from Syntrillium Software Corporation, Phoenix, Ariz.) digitized the sound waves into storage, manipulation, and output system 18, i.e., a computer file.

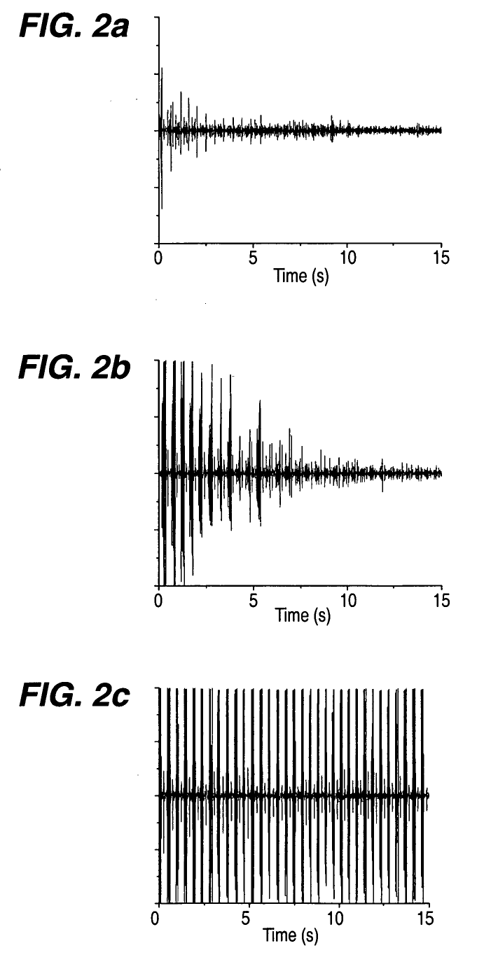

FIG. 2 shows the sound profiles of a common surfactant used in wash-off products, i.e., sodium PEG Lauryl ether sulfate (SLES)/water solution at different concentrations, generated using the system of FIG. 1. The observations from these Figures are:
(a) 1% SLES/water solution shows very low sound level all the time, indicating a slimy feel or tactile perception;
(b) 0.2% SLES/water solution shows the sound level from high to low as finger rubbing time goes on, indicating a transition from squeaky to slimy feel at this concentration;
(c) 0.01% SLES/water solution shows a very high sound level all the time, indicating a squeaky feel that does not goes away with time at this concentration.

The differences between these solutions are easily discernible from the results. For higher concentration (e.g. 1% SLES/water solution, FIG. 2a), the SLES film covers the entire skin surface and lubricates the two skin surfaces against their touching. No significant sound can be produced by rubbing fingers (sound pressure is very low). The consumer perceives sliminess of the solution.

With reference to FIG. 2(b), when the SLES concentration is lower (e.g. 0.2%), the sound profile is strongly dependent on rubbing time. In the beginning of rubbing, the sound level is very high, corresponding to a “squeaky” feel. As the rubbing continues, the sound level gradually decreases and the consumer can also perceive that the squeakiness decreases. When the solution is perceived as slimy, almost no sound is emitted.

Below a certain SLES concentration (FIG. 2c), the consumer can only feel squeakiness of solution no matter how long rubbing goes.

The results demonstrate a strong connection between the consumer tactile perceptions and acoustic emission signals. Different surfactant systems give different acoustic profile, thereby representing the different squeakiness/sliminess of those surfactant systems. The acoustic method can be used for quickly and simply evaluating surfactant systems for the tactile perception they produce.

### EXAMPLE 2

This example demonstrates an assessment of the rinse profile of wash-off products, simultaneously using hydrophone 22 and accelerometer 24.

With reference to FIG. 1, in this example, a typical set-up for assessment of wash-off (cleansing) products was used, employing simultaneously hydrophone 22 and accelerometers 24. To characterize the rinsability and “feel” of skin cleansers, the acoustic signal can be detected by hydrophone 22 immersed in a tank filled with water (not shown) for rinsing an area of skin. Two accelerometers 24 were attached to the subject's skin (PCB 352A24 accelerometer for normal vibration, and PCB 356A15 triaxial accelerometer for tangential and normal vibration). A known amount of cleansers was applied on the wet forearm with the hand of an individual. The arm was then immersed into the rinse tank full of certain hardness water at certain temperature.

The other hand rubbed the arm with product while the rubbing sound was picked by hydrophone 22 and skin vibration was detected by accelerometers 24, simultaneously.

The acoustic signal from hydrophone 22 was conditioned as in Example 1 and recorded by data acquisition system 16. Signals from accelerometers 24 were conditioned by PCB 442B104 signal conditioner 30 (PCB Piezotronics, Inc., Depew, N.Y.).

Data from data acquisition system 16 was communicated to storage, manipulation, and output system 18, i.e., a Cool Edit 2000 with sound card digitizing system. In addition, a professional acoustic system (Bruel & Kjaer Pulse 6.1 and 7.0, Atlanta, Ga.) was used.

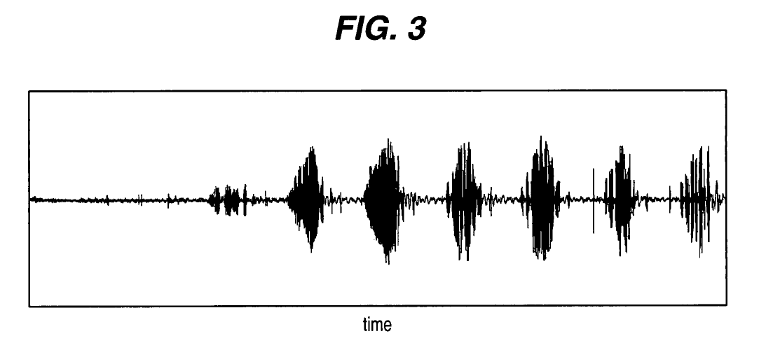

A rinse profile of a wash-off product (TEA-N-Lauroyl L-Glutamate (LT-12)) in soft water (40 PPM, Ca⁺⁺/MG⁺⁺=3/1) thereby obtained is shown in FIG. 3. The figure illustrates how, during rinse, the amplitude of the acoustic signal changes significantly. At first, because of the lubricating effect of the surfactants, the acoustic signal is very significantly reduced. The slimy region has no sound or very low sound level while the squeaky region has stronger sound level. As the rinse proceeds, much higher amplitude “squeaky” sounds are produced. The speed of rinse of the cleansers can be assessed on the wave file by measuring the time period before the occurrence of high amplitude squeaky sound. The squeakiness of the cleansers can be assessed by measuring the average amplitude of the squeaky sound. In this particular case, there is no acoustic emission from the first three rubs while consumer perceives the sliminess of the wash-off product. From the 4^{th} rub to 10^{th} rub, the acoustic emissions are different at each rub, indicating the squeakiness changes during rinse. In the 5^{th} and 6^{th} rubs, the more uniform sound profile relates to the smooth feel. In the last several rubs, there are larger interrupts between sound emission spikes, which correlate to the stick/slip of skin surfaces. The consumers usually perceive clean rinse when they reach these rubs.

The whole acoustic profile shows the very rich information during rinse of wash-off products. The different squeakiness can be observed from the sound profile and can be further extracted by applying different data analysis methods (e.g. Fast-Fourier transform (FFT) can reveal the frequency of stick/slip which relates to the squeakiness).

Moreover, the observed acoustic profile corresponds extremely well to the consumer's perceptions during rinse,

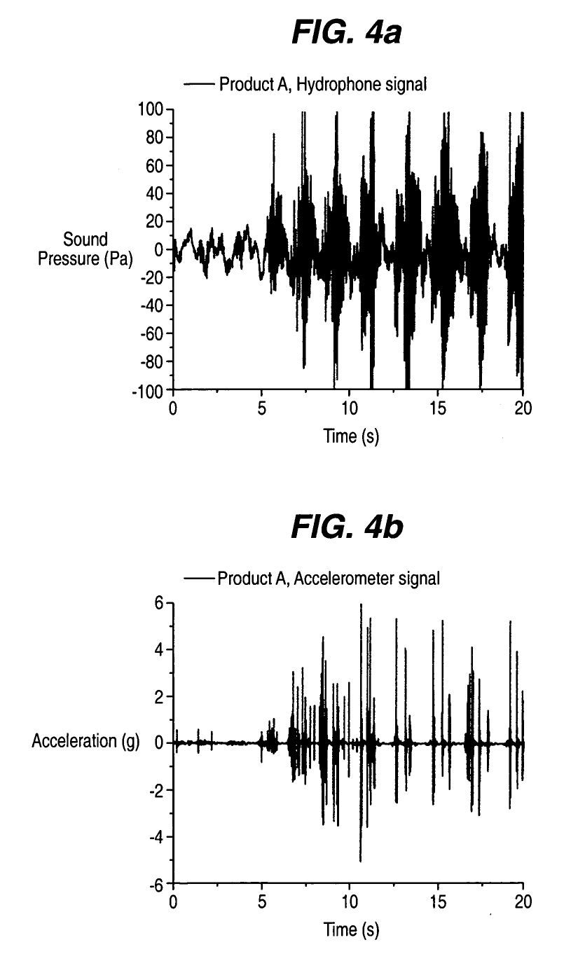

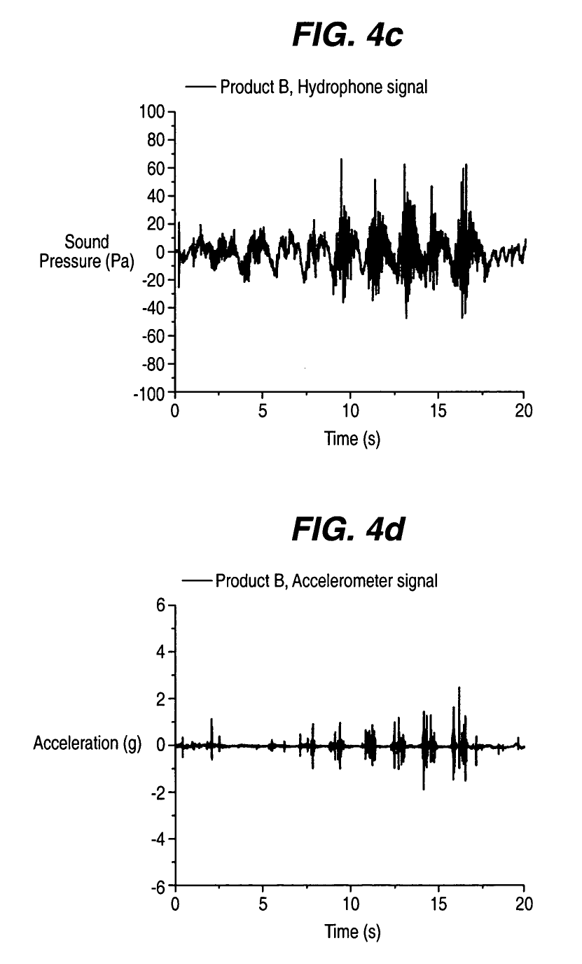

FIG. 4 shows two distinct rinse profiles obtained by rinse in soft water for two different products by acoustic methods using the combination of hydrophone 22 and accelerometers 24 as described in this Example. Product A is Kao White brand bar, a leading soap bar in the Japanese market. Product B is DOVE brand bar from Nippon Lever, Japan.

FIGS. 4(a) and 4(c) are signals from hydrophone 22 of two wash-off products. Product A has a quick rinse and feels very squeaky. As shown in FIG. 4(a), the slimy region lasts about 5 seconds when the sound pressure is low. In the squeaky region, the sound pressure is higher than 100 Pa. and fine analysis shows the very strong stick/slip signal. For product B, the slimy region lasts about 9 seconds and in the squeaky region, the sound pressure reaches peak value about 60 Pa. But most of signals remain under 45 Pa.

More clear differences between the products can be observed by comparison of skin vibration, as shown in FIGS. 4(b) and 4(d). Normal (to the skin surface) vibration of skin surface is shown. The acceleration shown in FIG. 4(b) can reach 6 g and the frequency of the vibration is medium at the beginning of squeaky region and is very low later, which indicates the strong stick/slip. Japanese consumers usually perceive clean rinse when they reach these rubs, and this it the preferred tactile perception for the Japanese market. However, American consumers perceive this tactile perception as harsh. In FIG. 4(d), the acceleration is much lower and the frequency is higher in the squeaky region, which was correlated to a perceived smooth feel.

The combination of hydrophone 22 and accelerometers 24 reveals the consumer perceptions during rinse and can be used as an instrumental tool for conducting consumer tests for wash-off products.

### EXAMPLE 3

20 This example demonstrates evaluation of sensory properties of skin care leave-on products.

With reference to FIG. 1, system 10 for acoustic measurement and evaluation of sensory properties of leave-on and wash-off products included two accelerometers 24 (PCB 352A24, PCB Piezotronics, Inc., Depew, N.Y.) attached to the subject's forearm.

One accelerometer 24 was attached near the palm of the hand and the other near the elbow, to sense the normal vibration of skin surface. Two microphones 20 (½′ type 4189 pre-polarized free-field microphone, Bruel & Kjaer, Atlanta, Ga.) were mounted above the two sites where accelerometers 24 were attached. To generate skin vibration, the subject used the other hand (fingers) to rub the forearm starting at the elbow and continuing in the direction of the palm of the hand. Alternatively, a motorized “hand” equipped with loading cell and friction sensor can also be used to rub instead of using human fingers, in order to control the loading, rubbing speed and measure the friction at the same time.

All acoustic signals were collected by Pulse data acquisition system 16 (Bruel & Kjaer, Atlanta, Ga.) and analyzed subsequent to collection by storage, manipulation, and output system 18.

In the following discussions, we only refer the signal from accelerometer 24 located closer to the elbow.

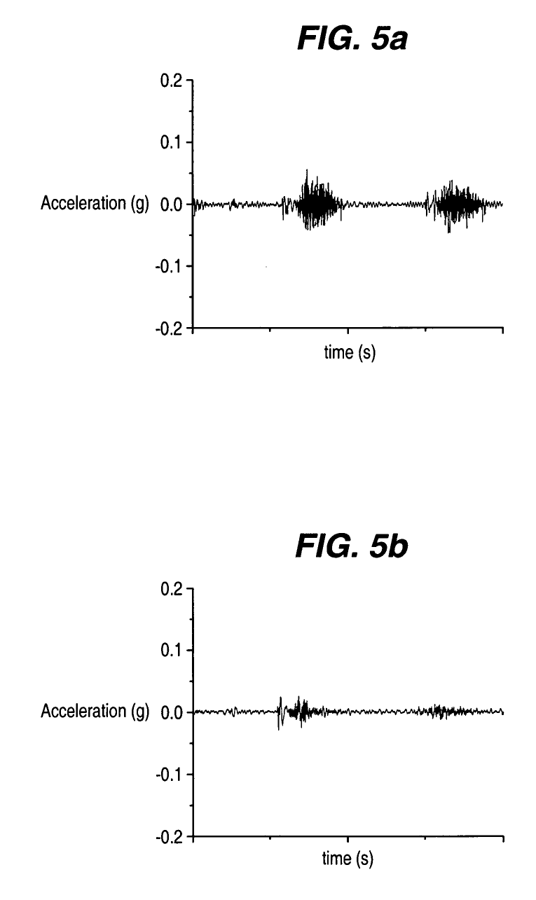

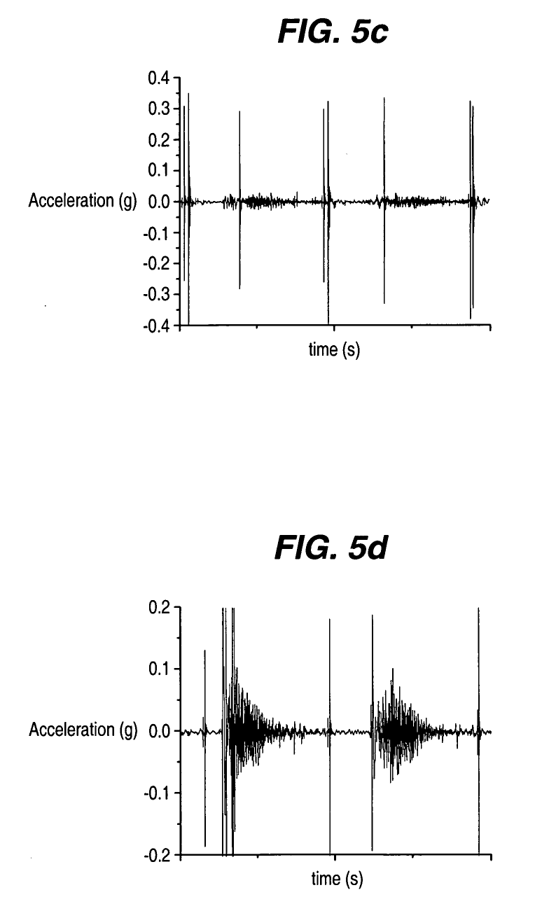

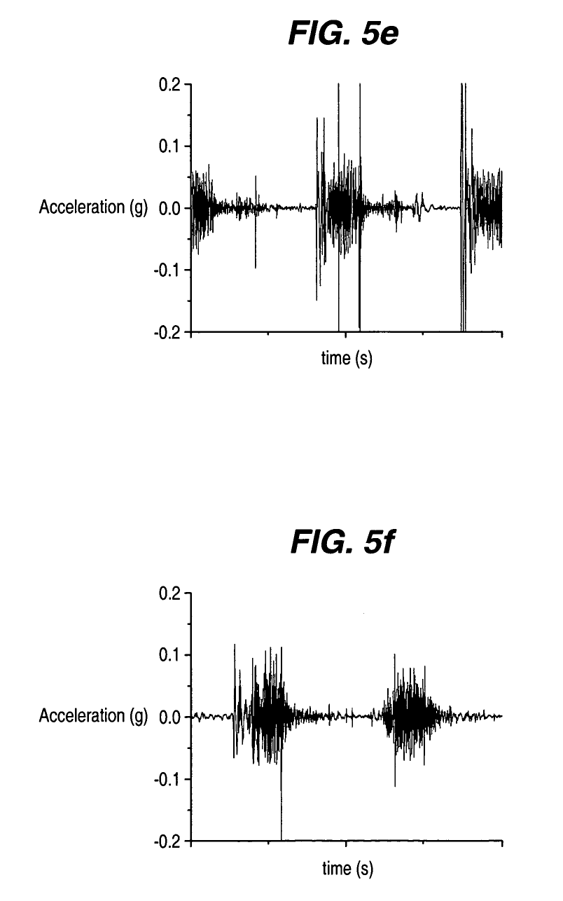

FIG. 5 shows the results of normal acceleration for before and after applying a skin care leave-on product to a subject's forearm. With reference to FIG. 5(a), before applying any skin care product, the skin vibrates in a certain band of frequency with certain amplitude. Fair & Lovely brand lotion, available from Hindustan Lever, a dry matte product specially formulated for the Indian market preference, was applied to the forearm. It was observed that after application of the skin care product, the skin vibration changed as the product dried. With reference to FIG. 5(b), while the product was still wet, the signal from accelerometer 24 showed very low amplitude vibration with lower frequency. With reference to FIGS. 5(c)-(e), during drying of the product, strong vibration peaks were observed. The strong vibration peaks related to the sudden releases of two stretched skin surfaces sticking together during sliding of fingers. At that stage, the subject felt strong dragginess of the product, which is preferred by the Indian consumer and other consumers in hot climates. FIG. 5(f) shows the acceleration of skin surface after the skin care product dried completely.

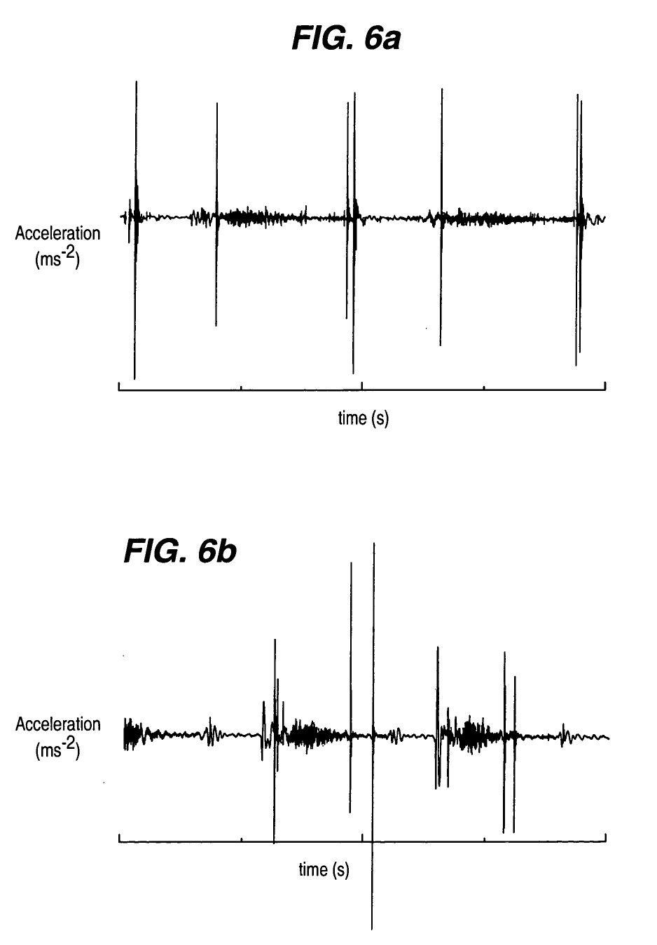

FIG. 6 demonstrates that different leave-on products create different feelings. The vibration detected via accelerometers 24 can differentiate those feelings very sensitively. FIG. 6 shows two acceleration curves of two different products which give consumer different feelings. The signals are displayed at the same scale for easy comparison. Product 1, Fair & Lovely brand cream, which feels dry and draggy, creates several strong vibration peaks which correlate to the stick-slip event of two sliding surfaces. Product 2, POND'S brand Age-defying Complex lotion (Chesebrough-Pond's, U.S.) produces an intermediate feel between smooth and draggy. Different consumers may prefer different tactile perceptions.

### EXAMPLE 4

This example demonstrates an assessment of after-use feel for wash-off products using accelerometers 24.

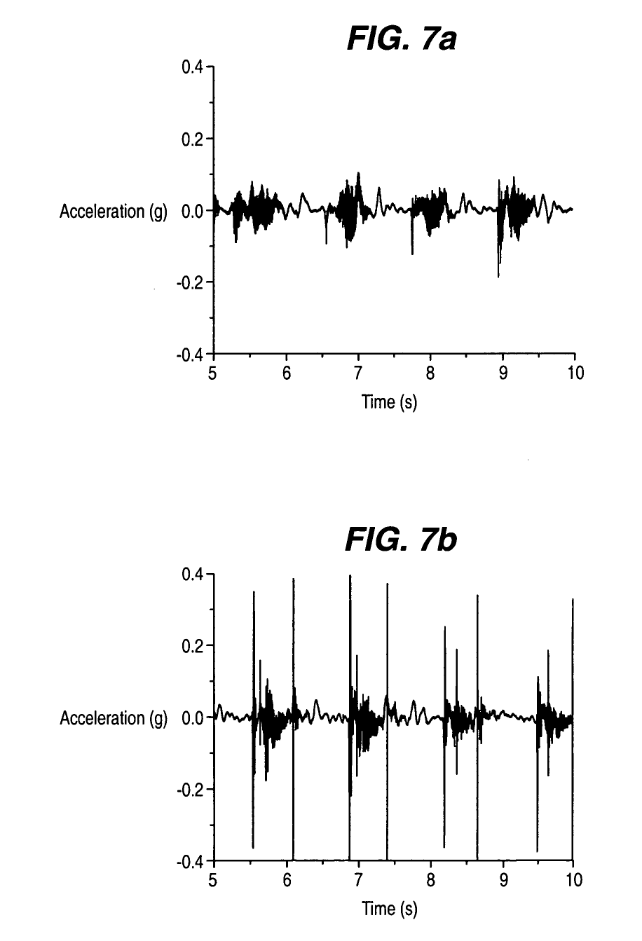

FIG. 7 shows two acceleration curves of after-feel. A consumer was asked to use two different cleansing products. After using certain amount of water for washing the products, the skin was patted dry with a tissue. The signals shown here were taken at 2 min after dry. FIG. 7(a) shows the after-feel curve of Kao White brand bar, which makes skin more hydrophobic and produces a “dry” feel. FIG. 7(b) shows the acceleration of a quite different cleansing product, DOVE brand bar, that makes skin hydrophilic and produces a moisturization feel. The strong stick/slip peaks were observed. The results show that the moisturization agents make the skin more tacky.

### EXAMPLE 5

The present invention is for objectively assessing attributes or condition of an area of human skin. Facial attributes, such as pores, wrinkles and photoaging, may be evaluated. The inventive system and method can be applied for consumer self-evaluation or for evaluation by a beautician or sales associate.

This example illustrates that an evaluation of pre- and post-treatment pore appearance is possible using acoustic emission system 10, suggesting the validity and usefulness of the system and method of the present invention.

Sheer Coverage brand foundation, available from Calvin Klein Cosmetics Co., New York, was evaluated using acoustic emission system 10. Good results were obtained. Consumers perceived a difference acoustic emission system 10 scale, as it correlated well with the visually perceived improvement in appearance of pores after application of the foundation.

### EXAMPLE 6

This example illustrates the use of acoustic emission system 10 and method as a consulting tool at point of purchase and/or as a tool for communicating with consumers.

Generally, consumers in Japan reject for purchase or use soap bars they perceive as “slimy”. A new soap bar, based on new technology, is developed and placed on the market in Japan. Acoustic emission system 10 and method are used to communicate to the Japanese consumers:
(a) that the new soap bar has changed tactile perception; and
(b) what will be different for the consumer in terms of sensory and end benefit.

Conversely, the product newly formulated for Japan would now be perceived as having an unpleasant “rub” by the American consumer, and a choice may be made available together with acoustic emission system 10 available at point of purchase to allow the consumer to select the bar that minimizes the unpleasant “rub.”

The method can be used as a communication tool to consumers (e.g. advertising).

### EXAMPLE 7

This example illustrates the use acoustic emission measurement system 10 and method for determining the condition of the skin pre- and post-treatment skin, and the usefulness of visual improvement in untreated skin condition to induce the consumer to continue using the product.

A consumer took a measurement of the pre-treatment condition of her facial skin using acoustic measurement system 10.

Subsequently, the consumer applied a POND'S Dramatic Results brand product, available from Chesebrough-Pond's, U.S., over a period of about four weeks. Another acoustic measurement was taken of clean facial skin without product application. The measurement indicated a significant improvement in appearance of facial skin wrinkles over the period of use.

Although the improvement after four weeks would not have been visually perceptible and would not have been perceptible to the touch, the improvement was evident from acoustic measurement, which encouraged the consumer to continue using the POND'S product.

Another advantage of the acoustic method of the present invention is that consumers cannot remember the condition of their skin before use. This method gives the consumers a way to compare the difference before and after use of cosmetic products. Therefore, acoustic emission measurements are a good tool for communicating with consumers regarding long term benefits of a given cosmetic product or regimen.

### EXAMPLE 8

Acoustic emission signal expected to result from use of a cleanser as shown in FIG. 2 was printed, folded into a concertina, or pamphlet and placed in a package containing the product.

### EXAMPLE 9

This example demonstrates the utility of acoustic emission system 10 and method to define consumer preferences.

During a focus group study, 10 consumers were asked to pick out a skin cleansing product that left them with their “ideal” end point skin after feel. The product selected by the majority of the consumers was evaluated using acoustic emission system 10 by the inventive method. With the emission profile in hand, different product formulations were evaluated to match the one preferred by the consumers.

Thus the inventive system and method served as a tool to generate purchase intent in consumers, as well as a tool for developing products to suit consumer preferences.

### EXAMPLE 10

This example discusses the use of acoustic emission system 10 and method in the development and validation of clinical scale.

In the consumer study discussed above, consumers were also asked to characterize each of the products tested as leaving a “slimy,” “smooth,” or “squeaky” tactile perception on the skin. This consumer data was correlated with acoustic emission signals for each of the products, to develop an emission scale that corresponds to consumer perceptions of slimy, smooth, or squeaky. The results of this exercise showed that the two grading methods are highly correlated with one another. The acoustic emission images were thus used as anchors to generate a reproducible clinical scale for the grading of tactile skin perception.

In a separate exercise, two clinicians verified that there was a high correlation between the consumer stated tactile perception and the corresponding acoustic signal image.

### EXAMPLE 11

This example demonstrates the utility of acoustic emission measurements according to the present invention in assisting with product selection.

POND'S Institute is set up in Spain, including a vending machine for personalizing leave-on and wash-off cosmetic products. Preparation of such products immediately upon demand has the advantages of custom products and is particularly advantageous for compositions including unstable ingredients which are best kept unmixed until close to time of use.

In this example, a consumer, with help from a beautician inputs personal preference information for a leave-on product (face cream). Additionally, an acoustic emission measurement was taken to determine the condition of her skin, such as moisture level, skin oiliness, etc.

The vending machine produces a custom cream based on the preference information input by the consumer, as well as based on acoustic emission measurements of her skin condition.

### EXAMPLE 12

This example demonstrates the utility of the acoustic emission measurement system and method for measuring tactile perception of “tacky.”

A consumer pushes and lifts fingers to and from a surface which can be described as “sticky,” “powdery,” etc. The skin vibrates during the pushing and lifting. Accelaration can be measures and used to monitor the feel.

The procedure of this example is particularly advantageous for deodorant products, where “tack” is a very important tactile perception.

### EXAMPLE 13

This example demonstrated the utility of acoustic emission system and method for measuring the cleanliness of hard surfaces. In this example, acoustic emission of skin touching glassware was measured.

The methodology of this example may be used to promote dish cleaning products.

While the present invention has been described herein with some specificity, and with reference to certain preferred embodiments thereof, those of ordinary skill in the art will recognize numerous variations, modifications and substitutions of that which has been described which can be made, and which are within the scope and spirit of the invention. It is intended that all of these modifications and variations be within the scope of the present invention as described and claimed herein, and that the inventions be limited only by the scope of the claims which follow, and that such claims be interpreted as broadly as is reasonable. Throughout this application, various publications have been cited. The entireties of each of these publications are hereby incorporated by reference herein.

1. An acoustic emission measurement system comprising:
(A) Means for generating an acoustic emission signal from skin;
(B) Means for collecting, storing and displaying said emission signal;
(C) means for correlating said emission signal with an attribute of said skin;
wherein said system is used as a clinical tool to evaluate efficacy of cosmetic skin care and/or cleansing products.

2. The system according to claim 1, wherein said means for displaying said emission signal comprises a medium selected from the group consisting of Internet, camera, palm pilot, mobile phone, mobile camera phone, and advertising and promotional material selected from the group consisting of television, magazines, brochures, posters, flyers, and hand-outs.

3. The system according to claim 1, wherein said system is used by a consumer, a beautician, a professional adviser, or combination thereof.

4. The system according to claim 1, wherein said correlation represents attributes of pores, wrinkles, photoaging, or skin texture.

5. A cosmetic product selection and/or customization system comprising:
(i) at least one cosmetic composition for reducing the appearance of at least one undesirable skin attribute; and
(ii) an acoustic emission system associated with said cosmetic composition;
the acoustic emission system having a means for evaluating current appearance of skin attributes or progress in reducing the appearance of said undesirable attributes with the use of said cosmetic composition.

6. The system according to claim 5, wherein said acoustic emission measurement system comprises a medium for indicia of at least two different skin attributes, thereby allowing consumers or clinicians to distinguish skin attributes resulting from application and/or wash-off of a cosmetic product.

7. The system according to claim 5, wherein said media are selected from the group consisting of Internet, camera, palm pilot, mobile phone and advertising material.

8. The system according to claim 5, whereby said system facilitates adherence by a consumer to a product usage regimen on the basis of said distinguished skin attributes.

9. The system according to claim 5, wherein said acoustic emission measurement system is placed into a carton alongside a container holding the cosmetic composition.

10. The system according to claim 5, whereby said system facilitates cosmetic product selection on the basis of said distinguished skin attributes.

11. The cosmetic system according to claim 5, wherein said facial attributes are selected from the group consisting of pores, wrinkles, photoaging, or skin texture.

12. A method for evaluating efficacy of a cosmetic product, the method comprising:
(A) providing a system according to claim 5;
(B) applying the cosmetic product to an area of skin;
(C) generating acoustic emission data for said area of skin;
(D) analyzing said data to assess said skin attributes; and
(E) repeating steps (C) and (D) at a future time followed by comparison of data resultant from first and second assessments of the skin.

13. The method according to claim 12, wherein said evaluation is a self-evaluation by a consumer or an evaluation by a clinician, beautician or sales assistant.

14. The method according to claim 12, wherein said assessments are of facial attributes are selected from the group consisting of pores, wrinkles, photoaging, and skin texture.

15. A method of evaluating facial attributes on an area of human skin of at least one individual, comprising:
(A) providing a system according to claim 1;
(B) generating acoustic emission data for said area of human skin;
(C) analyzing said data to define a distribution of skin attributes in a population of said individuals.

16. The method according to claim 15, wherein said facial attributes are selected from the group consisting of pores, wrinkles, photoaging, and skin texture.
